(19) United States
(12) Patent Application Publication
(10) Pub. No.: US 2023/0221970 A1
(43) Pub. Date: Jul. 13, 2023

(54) ANALYTICS DRIVEN USER GUIDANCE BASED ON USAGE DATA

(71) Applicant: JOHNSON & JOHNSON SURGICAL VISION, INC., Irvine, CA (US)

(72) Inventor: Mitchell Mallough, Irvine, CA (US)

(21) Appl. No.: 18/154,570

(22) Filed: Jan. 13, 2023

Related U.S. Application Data
(60) Provisional application No. 63/299,358, filed on Jan. 13, 2022.

(51) Int. Cl. G06F 9/451; G16H 10/20

(52) U.S. Cl. CPC G06F 9/453; G16H 10/20

(57) ABSTRACT

Methods and systems are provided. The methods and systems provide analytics driven user guidance based on data. The method and system are implemented by a determination engine stored on a memory as processor executable instructions. The methods and systems include receiving inputs associated with fields of a user interface and aggregating the data associated with the inputs. The methods and systems also include evaluating the data and the inputs to generate interface elements or confidence factors and providing the interface elements or the or more confidence factors, as the analytics driven user guidance, on the user interface to mitigate or prevent inadvertent errors.

### CROSS-REFERENCE TO RELATED APPLICATIONS

This application claims priority to U.S. Provisional Patent Application No. 63/299,358, filed Jan. 13, 2022, which is incorporated herein by reference in its entirety.

### FIELD OF INVENTION

The disclosure is related to systems and methods implementing analytics driven user guidance based on usage data.

### BACKGROUND

Medical information is a robust field that includes paper and electronic records for patients, medical/service providers, insurance companies, procedures, surgeries, etc. Conventional user interface designs are subject to user errors when entering, managing, and using medical information. There is a need to provide user guidance to medical professionals who enter, manage, and use medical information.

### SUMMARY

According to an embodiment, methods and systems are provided. The methods and systems provide analytics driven user guidance based on data. The methods and systems are implemented by a determination engine stored on a memory as processor executable instructions. The methods and systems include receiving one or more inputs associated with one or more fields of a user interface and aggregating the data associated with the one or more inputs. The methods and systems also include evaluating the data and the one or more inputs to generate one or more interface elements or one or more confidence factors and providing the one or more interface elements or the one or more confidence factors, as the analytics driven user guidance, on the user interface to mitigate or prevent one or more inadvertent errors within the one or more inputs.

According to one or more embodiments, the methods and systems can be implemented as an apparatus, a method, a system and/or a computer program product.

### BRIEF DESCRIPTION OF THE DRAWINGS

A more detailed understanding may be had from the following description, given by way of example in conjunction with the accompanying drawings, wherein like reference numerals in the figures indicate like elements, and wherein:

FIG. 1 illustrates a diagram of a system according to one or more embodiments;

FIG. 2 illustrates a diagram of a method according to one or more embodiments; and

FIGS. 3-18 illustrate diagrams of one or more user interfaces according to one or more embodiments.

### DETAILED DESCRIPTION

Disclosed herein are systems and methods for implementing analytics driven user guidance based on usage data. More particularly, disclosed herein are systems and methods implementing software (e.g., a determination engine) that procures and analyzes usage data respective to medical information and user actions to provide analytics driven user guidance that mitigates use errors. In this regard, the determination engine is processor executable code or software that is necessarily rooted in process operations by, and in processing hardware of, medical device equipment.

Generally, user interface designs are subject to use errors. Use errors can be categorized as deliberate errors and inadvertent errors.

Deliberate errors can include intentional non-compliant behavior that results in unusable data. To mitigate and/or prevent one or more deliberate errors, the determination engine can be configured to not let users perform any actions that they should not perform within one or more user interfaces.

Inadvertent error can include sub-optimal user experiences that results in sub-optimal data. Inadvertent errors can be further categorized as either action errors or thinking errors.

Action errors include when an action taken was not as planned, such as a user slip or a user lapse. A slip can include a typo within a data entry field, such as typing a “4” where a “5” was intended). A lapse can include a memory-based error, such as when a user forgets to enter any value for a field.

Thinking errors include when a planned action is accompanied by a mistake, such as a rule-based mistake or a knowledge-based mistake. A rule-based mistake can include when a rule or procedure is mistakenly applied to a wrong circumstance, such as transposing values for related entries (e.g., transposing Steep K and Flat K values). A knowledge-based mistake can include when no rules or procedures are in place and the planned action is based on a misinterpretation, such as when a user is not familiar with surgically induced astigmatism (SIA) and mistakenly attempts to enter preoperative astigmatism instead. To mitigate and/or prevent one or more inadvertent errors, the determination engine can be configured to provide to the user labeling, contextual help, data entry validation, messages, and/or warnings within one or more user interfaces.

Further, some inadvertent errors may not be mitigated or prevented by configurations, such as inadvertent errors of tolerant inputs and tolerant selections. Tolerant inputs can be entry errors, such as data entries that fall within acceptable data entry ranges while still representing a mistaken input. Tolerant selections can be selections that are not incorrect but are sub-optimal to other results.

According to one or more embodiments, the determination engine implementing analytics driven user guidance based on usage data from previous users to mitigate and/or prevent inadvertent errors of tolerant inputs and tolerant selections. Analytics driven user guidance can be implemented by the determination engine using machine learning and/or artificial intelligence (ML/AI). Analytics driven user guidance can also include the determination engine leveraging “big” data analysis, collective data analytics, one or more software models (e.g., Software as a Medical Device (SaMD) applications and/or medical device applications) and/or web-based calculators (e.g., Toric calculator, limbal relaxing incisions (LRI) calculator, etc.). For example, by applying the collective data analytics associated with a given SaMD application, the determination engine can use data entries and data selections to provide additional guidance to the user to prevent tolerant inputs (e.g., entry errors) and ensure informed selections (e.g., selections made based on as much information as possible). Thus, one or more technical effects, advantages, and benefits of the determination engine include eliminating data entry burdens and transcription error risks.

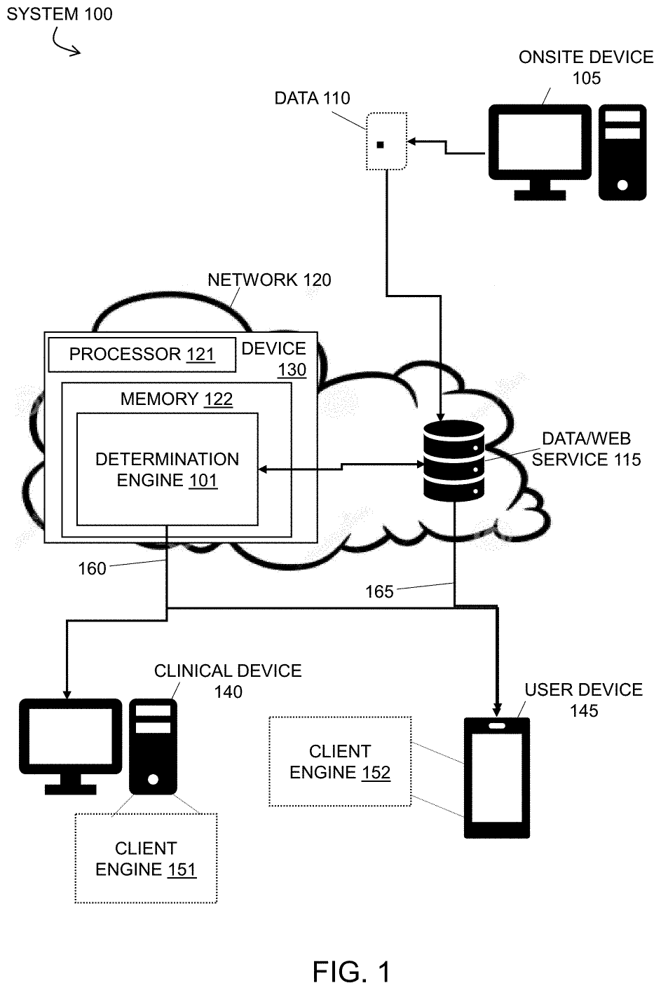

FIG. 1 is a diagram of a system 100 including a determination engine 101 according to one or more embodiments. All or parts of the system 100 and/or the determination engine 101 may be used to automatically implement analytics driven user guidance to mitigate and/or prevent deliberate errors and/or inadvertent errors. More particularly, all or parts of the system 100 and/or the determination engine 101 can make a binary determination as to whether the determination engine has enough information to perform a calculation. Further, all or parts of the system 100 and/or the determination engine 101 can determine as to whether the determination engine has proper information to perform the calculation and can fix any entry errors thereof. Further, all or parts of the system 100 and/or the determination engine 101 can provide subtle adornments/augmentations to one or more user interfaces (e.g., so things stay familiar to the users) and can compensate for translation needs.

As shown, the system 100 includes an onsite device 105 that outputs data 110 to at least a data/web service 115 of a network 120 for storage. The system 100 also includes a device 130 that depicts a processor 121 and a memory 122 storing the determination engine 101 thereon. The system 100 also includes a clinical device 140, a user device 145, each of which can include client engines 151 and 152 thereon. Note that, while single elements of the system 100 are shown, these elements represent one or more of that element. Each element of the system 100 is now further described.

The determination engine 101 and the client engines 151 and 152 can generally be viewed processor executable instructions. The determination engine 101 may be considered a determination engine software server. The determination engine 101 may employ one or more of artificial intelligence, modeling, machine learning algorithms, and clinical calculation algorithms. The client engines 151 and 152 acts as a client software instance of the determination engine 101. In this regard, the client engines 151 and 152 can mirror capabilities of the determination engine 101, while offloading processing responsibility.

According to one or more embodiments, the determination engine 101 implements analytics driven user guidance based on the data 110 respective to one or more user interfaces. Note that aspects/logic of the determination engine 101 can be incorporated into any user interfaces downloaded and presented at the clinical device 140 and/or the user device 145. As shown by example in FIG. 1, the client engines 151 and 152 can download (see arrows 160) one or more aspects/logic of the determination engine 101. Also, the client engine 152 can directly interact (see arrow 165) with data 110 of the data/web service 115 (e.g., in real time). In either case, the client engines 151 and 152 can access uniform resource locator (URL) of the determination engine 101 to load client side. According to one or more embodiments, the determination engine 101 implements and the client engines 151 and 152 can implement and/or interact with one or more devices, calculators, algorithms, etc.

According to one or more embodiments, the determination engine 101 can utilize data entries of the data 110 to provide additional guidance based on a normal distribution of values for a specific entry or combination of entries. In this regard the data entries can include published statistics. Further, to account for shifts in population and technology, the data entries can include refined statistics based on data analytics collected over time by the determination engine 101. For example, though a range of inputs for an axial length of an eye may vary from twelve (12) millimeters to forty (40) millimeters, a number of actual patients over age five (5) with an axial length below eighteen (18) millimeters is low (e.g., over the age of five (5), it is increasingly unlikely that a patient would have an axial length less than eighteen (18) millimeters). Based initially on published statistics of the data 110 and/or data analytics collected over time, an entry of fourteen (14) for the axial length in a patient of age sixty-five (65) can be identified/flagged by the determination engine 101 as a possible data entry error where the user meant to enter twenty-four (24). The determination engine 101 can also leverage relationships between keratometry (e.g., measuring curvature of the cornea) and biometry (i.e., measuring cornea power and eye length) inputs and the other inputs for patient age and calculation preferences to produce “confidence” factors for various inputs.

According to one or more embodiments, the determination engine 101 can utilize result selections of the data 110 to provide additional guidance based on a normal distribution of values for selections by previous users. For instance, calculation results for one or more web-based calculators leveraged by the determination engine 101 can display different lens models (e.g., up to three models) in one or more user interfaces. Expected residual astigmatisms produced by these lens models and presented in the one or more user interfaces, as well as whether axis flip varies, can influence a user's decision to choose a given lens model. According to one or more technical effects, advantages, and benefits, the determination engine 101 shows a user through the one or more user interfaces that previous users overwhelmingly chose a specific lens model or overwhelmingly avoided a specific lens model, so that the user has additional information when making a choice. Further, the determination engine 101 can indicate a number of similar calculations where no result was selected, indicating that none of the lens models shown are a good candidate (e.g., when a combination of preoperative corneal astigmatism and surgically induced astigmatism result in an astigmatism that cannot be adequately addressed by an available lens models available (e.g., such as a combined astigmatism of 4.5 diopters where a chosen lens family does not offer extended cylinder range models).

According to one or more embodiments, the determination engine 101 can implement range checking for data entry validation and can implement confirmation messaging for output selection. According to one or more embodiments, the determination engine 101 can use corroborating data and data analytics to identify and help to prevent other potential entry or selection issues.

Usage data is represented as the data 110, which can be any information respective to medical information and user actions that is utilized by the determination engine 101. More particularly, the data 110 can include any activity, interaction, or manipulation of one or more user interfaces (e.g., internet browsers, graphical user interfaces, window interfaces, and/or other visual interfaces for applications, operating systems, file folders, and the like), as well as the information submitted and/or generated by the one or more user interfaces. Examples of the data 110 include information collected during the course of ongoing patient care in varying mediums/forms, such as electronic health records or patient profiles. Further, the data 110 can include published statistics, administrative data, claims data, patient/disease registries, health surveys, clinical trial data, etc.

According to one or more embodiments, the data 110 includes diagnostic and/or clinical data for at least vision care, as well as refined diagnostic information based on any raw patient data that is entered by a medical professional (e.g., technician, nurse, surgeon, etc.). Data 110 examples of diagnostic and/or clinical data for vision care include, but are not limited to, eye dimension information and other physical characteristics of the eye (mapping out multiple indexes of the eye or mapping), ocular characteristics or anatomy, prescription information, eye disease information, eye disease symptoms, cataract information, glaucoma information (e.g., intraocular pressure), dry eye information, surgery system data, calculator information, ranges and percentages, and the like. Eye dimension information and/or ocular characteristics or anatomy can include, but are not limited to, keratometry and biometry, such as ocular biometry information, anterior corneal surface information, posterior corneal surface information, anterior lens surface information, posterior lens surface information, lens tilt information, and lens position information. Data 110 examples of diagnostic and/or clinical data for vision care can also include information regarding custom intraocular lenses, custom contact lenses, custom corneal implants, and the like, which can be configured to treat or ameliorate any of a variety of vision conditions in a particular patient based on their unique ocular characteristics or anatomy. Data 110 examples of surgery system data include, but are not limited to, alternative eye treatment procedure data, spectacle lens information, intraocular lens information, contact lens information, corneal ring implant information, collagenous corneal tissue thermal remodeling information, corneal inlay/onlay information, and corneal implant or graft information, along with parameters related to dioptic power, refractive index, anterior and posterior radius, lens thickness, asphericity, toricity, echelette design, haptic angulation, and lens filter. Further, examples of surgery system data include, but are not limited to various degrees of intraoperative rotation/tip/tilt associated with implantation of an intraocular lens and/or a variety of optical treatment modalities, along with vision treatment shapes or designs that can be administered to a patient. Thus, the data 110 contemplates a variety of vision related data, treatments, diagnostic data, and measurements that can be used by the system 100 to analytics driven user guidance.

According to one or more embodiments, the data 110 can include data entries and/or result selections, such as those provided by the onsite device 105. These data entries can be used by the determination engine 101 provide additional guidance based on a normal distribution of values for a specific entry or combination of entries, and/or selections by previous users.

According to one or more embodiments, the onsite device 105, the device 130, the clinical device 140, user device 145, and/or the data/web service 115 can structurally be any computing device comprising software and/or hardware, such as a general-purpose computer, with suitable interface circuits for transmitting and receiving signals (e.g., the data 110) to and from other items of the system 100. The hardware of these devices can include at least one processor and at least one memory, both of which are represented by way of example by the processor 121 and the memory 122. The processor 121 is representative of any computing circuit, central processing unit, microprocessor, and/or the like. The memory 122 is any non-transitory tangible media, such as magnetic, optical, or electronic memory (e.g., any suitable volatile and/or non-volatile memory, such as random-access memory or a hard disk drive).

The memory 122 stores processor executable instructions (e.g., of the determination engine 101) for execution by the processor 121, as well as the data 110 as needed. The processor 121, in executing the processor executable instructions, can be configured to receive, process, and manage the data 110, as well as communicate the data 110 to the memory 122 for storage and/or across the cloud environment 115.

Examples of the onsite device 105 include medical diagnostic equipment and a hospital workstation, as well as other diagnostic, therapeutic, and surgical devices. Medical diagnostic equipment can include, but is not limited to, auto analyzer machines, optical coherence tomography (OCT) devices, laser interferometers, corneal topography devices, phacoemulsification machines, corneal tomography devices, each of which can generate the data 110 pre-, intra-, and post-operatively. By way of example and representation, the onsite device 105 can be a CATALYS™ Precision Laser System by Johnson & Johnson Surgical Vision, Inc.

Examples of the device 130 include any server or computing system that provides input/output (I/O) communication interfaces that enables receiving signals from and/or transferring signals to other devices, such as by utilizing any number and combination of networks and various communication technologies, as described herein. The device 130 can be easily scalable, extensible, and modular, with the ability to change to different services or reconfigure some features independently of others.

Examples of the clinical device 140 include a stationary or standalone computer processor, a desktop or laptop computer, a hospital workstation, medical diagnostic equipment, surgical tools, Internet of Things (JOT) devices, etc., which can include modems, router capabilities, and/or any number and combination of networks and various communication technologies.

Examples of the user device 145 include a mobile phone, a smart phone, smartwatch, tablet or other portable smart device, which can include a camera and a display.

The data/web service 115 can be any database or computing system that stores an organized collection of structured and/or unstructured information (i.e., the data 110). For instance, the data/web service 115 can be a cloud-based clinical data repository of the data 110 that supports centralized and/or distributed processing of the device 120 and the determination engine 101. According to one or more embodiments, the data/web service 115 can be co-located with the device 130.

The network 120 may be a wired network, a wireless network, and/or include one or more wired and wireless networks, such as an intranet, a local area network (LAN), a wide area network (WAN), a metropolitan area network (MAN), a short-range network, a direct connection or series of connections, a cellular telephone network, or any other network or medium capable of facilitating communication between the items of FIG. 1 using any one of various communication standards/protocols (e.g., Bluetooth, Wi-Fi, Zigbee, Z-Wave, near field communications (NFC), infrared (IR), Ethernet, Universal Serial Bus (USB), or any other communication standards/protocols). Additionally, several networks may work alone or in communication with each other to facilitate communication in the network 120. In some instances, the device 130 and/or the data/web service 115 may be implemented as a single physical server on the network 120. In other instances, the device 120 and/or the data/web service 115 may be implemented as a virtual server on a public cloud computing provider (e.g., Amazon Web Services (AWS)®) of the network 120.

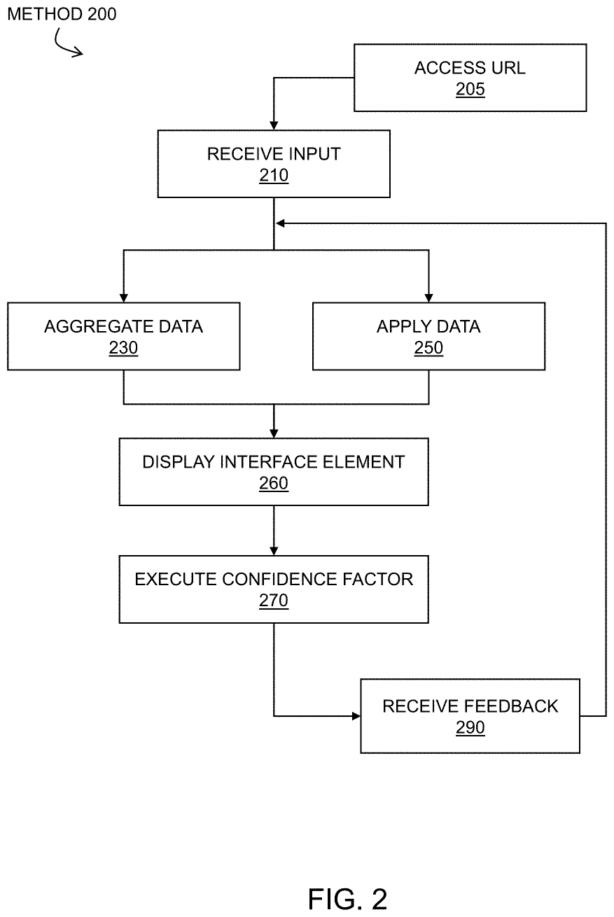

Turning now to FIG. 2, a diagram of a method 200 is shown according to one or more embodiments. The method 200 describes an example of analytics driven user guidance operations implemented by the determination engine 101.

Generally, the method 200 can be implemented for user input validation and output selection guidance to address inadvertent errors attributable to action errors and thinking errors. The method 200 can begin at block 205, where a URL of the determination engine 101 is accessed. According to one or more embodiments, accessing the URL of the determination engine 101 can enable the operations of the determination engine 101 to be presented in a web-based format with a user interface in a web browser viewed on the user device 145.

At block 210, the user interface of the determination engine 101 receives one or more inputs. The one or more inputs can be user inputs associated with fields of the user interface. Note that the user interface can be generated and presented by the determination engine 101.

At blocks 230 and 250, the determination engine 101 can aggregate and/or evaluate the data 110. These operations can occur in parallel as shown or sequentially. Aggregation and evaluation enable the determination engine 101 to generate one or more interface elements and one or more confidence factors.

Further, the determination engine 101 can operate in an offline mode or a live mode. In the offline mode, the aggregation and evaluation of the data 110 occurs after the determination engine 101 received the one or more inputs and/or during background/overnight processing. In this way, analytics after the fact can support decision making based on thresholds found in the data 110. In the live mode, the aggregation and evaluation of the data 110 in real time, such as while the medical professional is performing a procedure on a patient and providing the one or more inputs. In this way, real-time analytics can support decision making based on confidence factors.

At block 260, the determination engine 101 can provide/display the one or more interface elements, such as on the user interface. The one or more interface elements can be any annotation, indication, or the like that provides information and/or a warning. Examples of interface elements include, but are not limited to, geometric shapes, boxes, rectangular boxes, circles, ovals, highlighted areas, grey-out areas, bolded/underlined/italicized texts, modified texts, symbol, icon, and arrows, as well as color coding. Other examples of the one or more interface elements are further discussed herein.

The determination engine 101 can also provide/display the one or more confidence factors on the user interface, such as on the user interface. The one or more confidence factors can be any annotation, indication, or the like that provides information and/or a warning. Examples of confidence factors include, but are not limited to, alpha-numeric values, shapes, objects, colors, and combinations thereof on a range of no confidence to full confidence. Each confidence factor can be selected from the range and provided to show a confidence level in the one or more user inputs. Other examples of the one or more confidence factors are further discussed herein (e.g., confidence factors discussed in the context of representations through percentages, numbers, etc.).

At block 270, the determination engine 101 can apply the one or more confidence factors. In this regard, the determination engine 101 can apply the one or more confidence factors based on a variety of inputs and selections across numerous SaMD applications and medical device applications and can extend to connected equipment or IOT devices.

At block 290, the determination engine 101 can receive user feedback. The user feedback can include additional and/or alternative inputs into the one or more user interfaces, which can be further aggregated and evaluated by the determination engine 101. For instance, a user can change an input provided at block 210 based on a displayed interface element and confidence factor. This changed input can then be evaluated, and one or more new/subsequent interface elements and confidence factors can be provided as the determination engine 101 loops through the method 200.

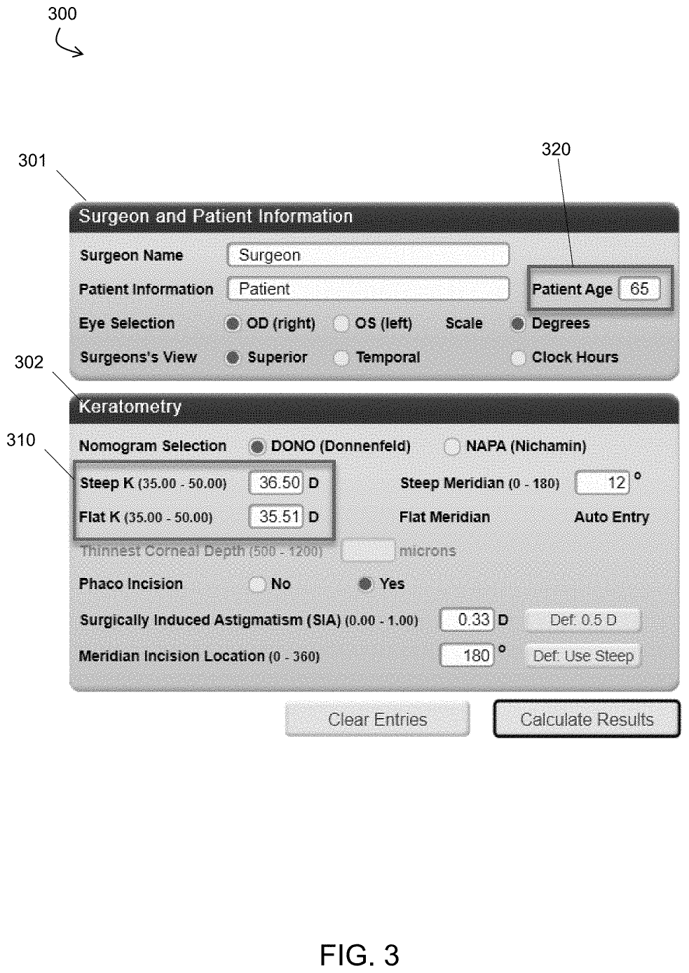

Turning now to FIGS. 3-17, diagrams of one or more user interfaces are depicted according to one or more embodiments. More particularly, the diagrams of FIGS. 3-17 describes and outlines examples of action errors and thinking errors, as well as provides examples across different SaMD and medical device applications, to illustrate the one or more technical effects, advantages, and benefits to users and patients.

FIG. 3 depicts a user interface 300 according to one or more embodiments. The user interface 300 relates to action errors and includes sub-interfaces 301 and 302. The user interface 300 can be a graphical user interface of an LRI calculator showing likely improper entries for flat K1 and steep K2 in box 310. Note these improper entries can also be shown with respect to a toric calculator or an online toric calculator.

Generally, the determination engine 101 can catch action errors, such as lapses (e.g., forgetting to enter or select a value), by applying acceptable ranges (identified by aggregating and evaluating the data 110) to various inputs and determining if the various inputs are within that range. Further, the determination engine 101 can also catch action errors, such as slips (e.g., typos or improper selections), that are within acceptable ranges and are not intended. As seen in the user interface 300, the input values for the steep K and the flat K in the LRI calculator are within the acceptable range of 35.00 diopters to 50.00 diopters.

According to one or more embodiments, the determination engine 101 utilized the data 110 (e.g., data analytics from previous calculations performed by all previous users) to determine a range of K values for an adult population. In turn, the determination engine 101 determines that 90% of K values (e.g., a sum of the steep K and flat K divided by two) for an adult population fall within a range of 40.5 diopters to 46.5 diopters. Note that, in some cases, the determination engine 101 can intelligently pull some of the data 110 (e.g., using a data lake), rather than all the data 110, to save processing overhead. In this way, the determination engine 101 can intelligently target information based on what calculation is being performed.

Further, the determination engine 101 determines that, for this set of entries, the K value is 36.01 (e.g., (36.50+35.51)/2=36.01). Further, since a patient age is sixty five 65, as identified in box 320, the determination engine 101 determines that the patient is within the adult population. Thus, because 90% of the K values for the adult population fall within the range of 40.5 diopters to 46.5 diopters, the K value for the set in box 301 is identified by the determination engine 101 (using one or more interface elements) as having a strong possibility/probability that the user intended to enter 46.50 and 45.51. Note that the boxes 310 and 320 can be examples of the one or more interface elements.

With respect to the strong possibility/probability, the determination engine 110 can corroborate one or more confidence factors with the user interface 300. For instance, a percentage of previous calculations where the K value of 36.01±2 in an adult patient was 0.01% (1 in 10,000 patients) would yield a low confidence factor from the determination engine 110 as discussed herein. Additionally, when in an offline mode, the determination engine 110 can update established ranges and percentages by harvesting and analyzing newly acquired data 110. For example, on a monthly basis, a toric calculator can provide at least 15,000 to 30,000 calculations from 10,000 users, and LRI calculator can provide at least 5,000 to 10,000 calculations from 5,000 users, which can total over 20,000 to 40,000 calculations per month (e.g., 0.5 million calculations per year). Thus, after at least one month, the established ranges and percentages may vary based on the additional calculations.

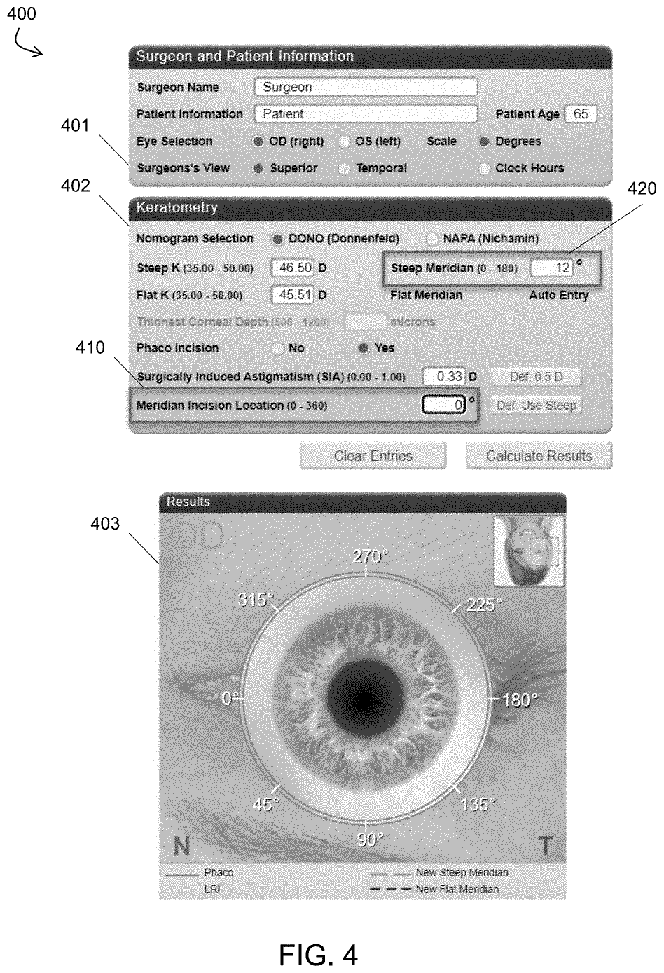

FIG. 4 depicts a user interface 400 according to one or more embodiments. The user interface 400 relates to thinking errors and includes sub-interfaces 401, 402, and 403. The user interface 400 can be a graphical user interface of an LRI calculator showing likely improper entries for meridian incision location in box 410. Generally, the determination engine 101 can catch thinking errors, such as where a user has a false memory or has incomplete knowledge and attempts to make an entry or selection based on their best understanding of a situation (e.g., the end user who “thinks they understand” or is making “their best guess” and acts on improper understanding). The determination engine 101 can also label the one or more user interface and/or provide contextual help and can corroborate one or more confidence factors with the user interface 400 to prevent thinking errors. Note that thinking errors are extremely difficult to detect.

As seen in the user interface 400, the input values for the steep meridian and the meridian incision location in the LRI calculator are within the acceptable ranges of (0-180) and (0-360) respectively. As seen in sub-interface 403, a meridian incision location of 0 degrees for an OD (right) eye places a primary incision on a nasal side of the eye and would be extremely difficult to perform without imparting significant additional surgically induced astigmatism. Regardless of whether this is a false memory that 0 degrees is temporal, there was confusion about the selected eye or is a gap in knowledge of the end user. Further, based on the location of the steep meridian, there are ample opportunities to locate the meridian incision location more preferentially temporal. In turn, the determination engine 101, based on the steep meridian and meridian incision location provided in boxes 410 and 420, can provide a low confidence factor for the eye selection and the meridian incision location. Note also that these thinking errors can also be shown with respect to a toric calculator or an online toric calculator.

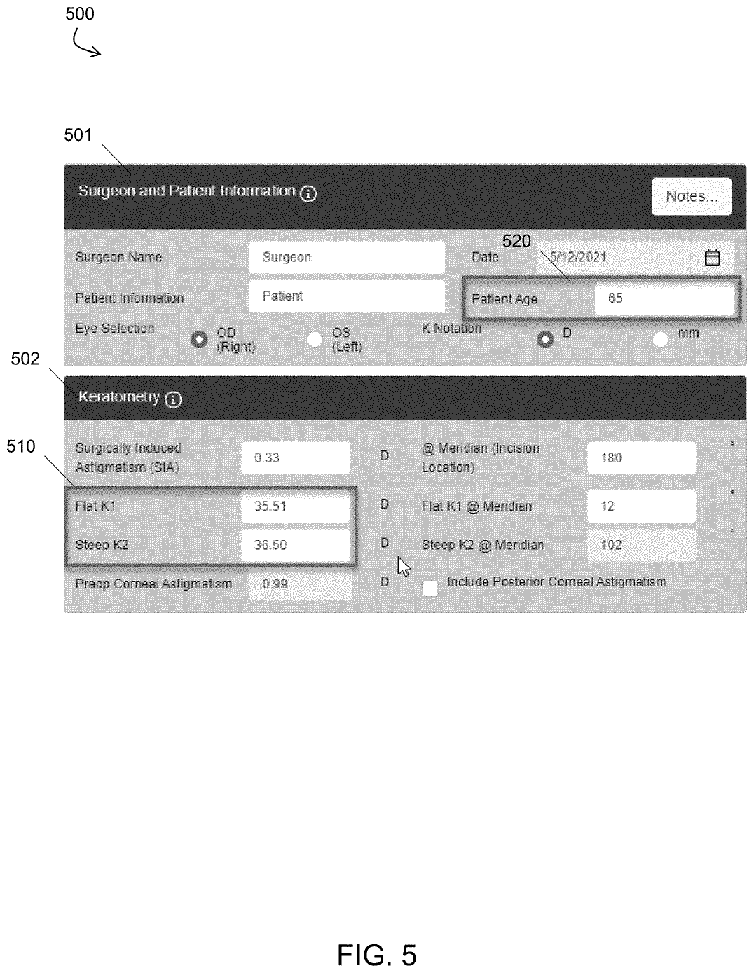

According to one or more embodiments, the determination engine 101 can provide additional confidence factors for input validation. In this regard, the confidence factors can be applied based on a variety of inputs and selections across numerous SaMD applications (and/or medical device applications) and can even be extended to connected equipment or devices (e.g., classified as IOT devices). More particularly, FIGS. 5-9 are examples of the determination engine 101 providing input validations associated with a toric calculator or an online toric calculator. Note that in the examples of FIGS. 5-9, all extreme cases would likely result in a low confidence factor by the determination engine 101 regardless of the use of data analytics, as data analytics would be more of a factor in subtle cases where a result is less likely or improbable but not impossible.

FIG. 5 depicts a user interface 500 according to one or more embodiments. The user interface 500 relates to double entry error and includes sub-interfaces 501 and 502. The user interface 500 can be a graphical user interface of the toric calculator showing likely/probable entry error for flat K1 and steep K2 in box 510. Note the determination engine 101 can verify that the patient is within an adult population based on the patient age of box 520. In this way, the determination engine 101 can provide a low confidence factor in any situation where the K value is not in alignment with the patient age.

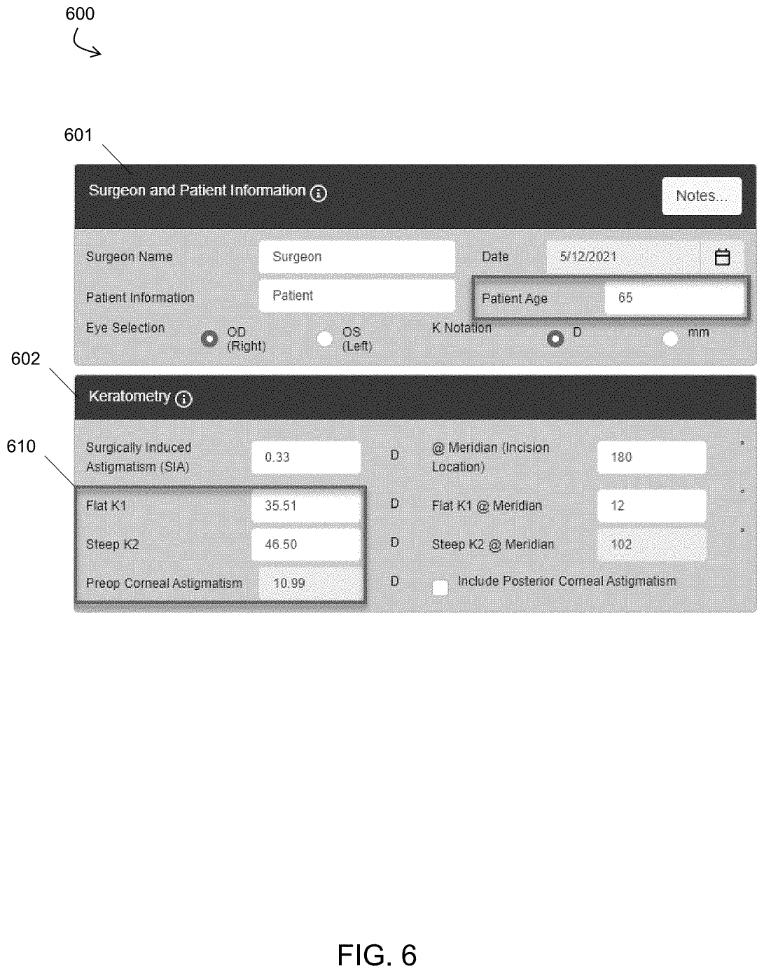

FIG. 6 depicts a user interface 600 according to one or more embodiments. The user interface 600 relates to single entry error and includes sub-interfaces 601 and 602. The user interface 600 can be a graphical user interface of the toric calculator showing likely/probable entry error for flat K1 in box 610. Note that the determination engine 101 can provide an additional input validation that identifies an action error for either the flat K or the steep K that results in an unusually large preop corneal astigmatism. For instance, as shown in the user interface 600, the user may have accidentally entered a flat K of 35.51 diopters (as the user likely meant to enter a flat K of 45.51 diopters with a steep K of 46.50 diopters). While, what is entered in box 510 is still a valid value, the result is an extremely large preop corneal astigmatism of 10.99 diopters. In a subtler case, the typo for the flat K can be in a second digit instead of a first (e.g., 43.51), which may still cause the determination engine 101 to produce a low confidence factor using data analytics.

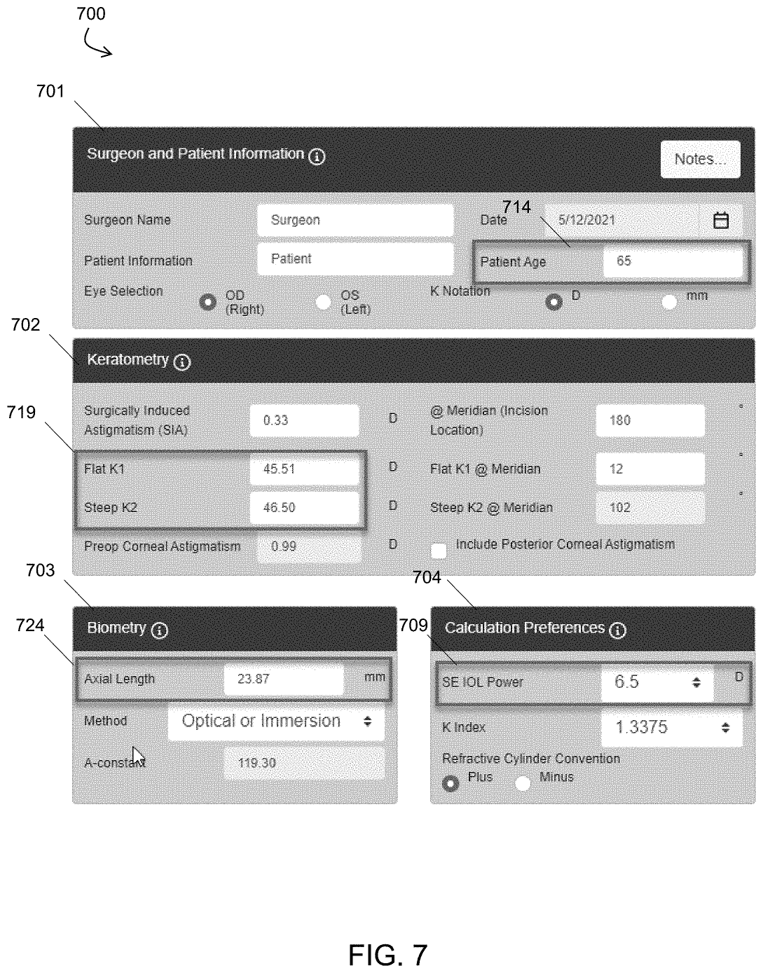

FIG. 7 depicts a user interface 700 according to one or more embodiments. The user interface 700 relates to improper entry selection and includes sub-interfaces 701, 702, 703, and 704. The user interface 700 can be a graphical user interface of the toric calculator showing likely/probable selection error for SE interocular lens (IOL) power in box 709. According to one or more embodiments, the determination engine 101 can provide input validation for an SE IOL power action error (e.g., improper selection due to mouse or keyboard processing) and/or thinking error (e.g., improper selection due to misunderstanding). That is, based on aggregating and processing the data, the determination engine 101 determines that the SE IOL power selection can range from 5.0 diopters to 34.0 diopters (in half-diopter increments). Further, the determination engine 101 determines that a typical power of the lens of the human eye is from about 14.5 diopters to 25.5 diopters dependent upon K value and axial length. As seen in the user interface 700, an adult patient (based on the patient age of box 714) with a K value of 46.01 diopters (based on the flat K1 and the steep K2 of box 719) and a rather typical axial length of 23.87 millimeters (see box 724) is extremely unlikely to have an SE IOL Power of 6.5 diopter. In turn, the determination engine 101 can flag/identify the 6.5 diopter with a low confidence factor. Data analytics of the determination engine 101 could further refine this low confidence factor for SE IOL power selections when the selection issue is not as extreme as well.

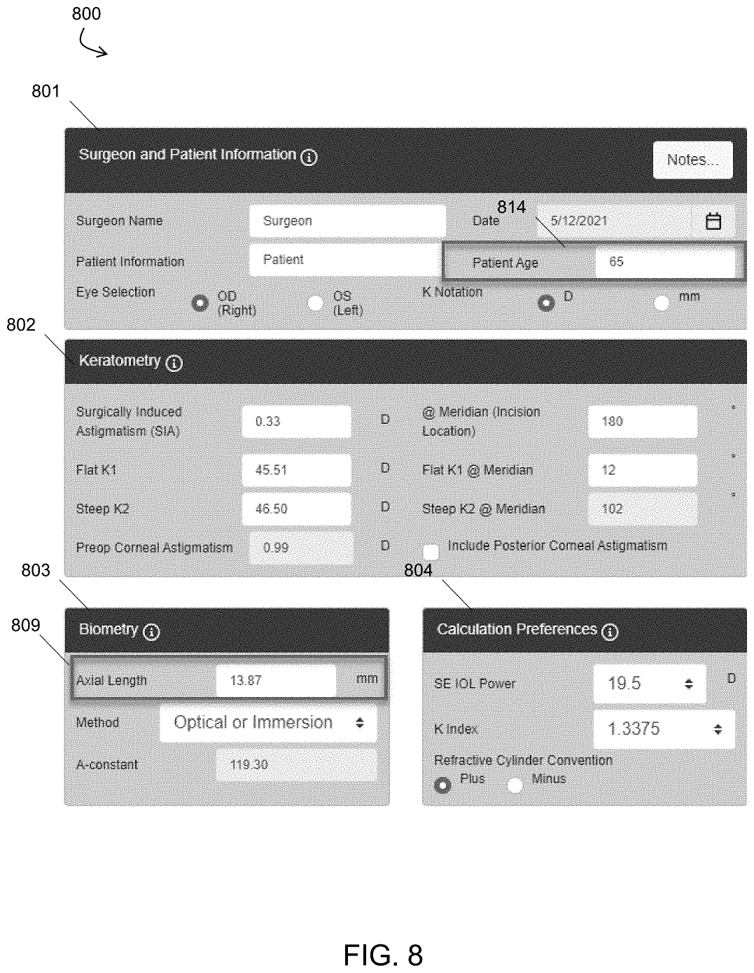

FIG. 8 depicts a user interface 800 according to one or more embodiments. The user interface 800 relates to single entry error and includes sub-interfaces 801, 802, 803, and 804. The user interface 800 can be a graphical user interface of the toric calculator showing likely/probable entry error for axial length in box 809. The error in box 809, when a patient is clearly an adult (based on the patient age of box 814), is suggestive that the user entered 13.87 millimeters when 23.87 millimeters may have been intended. In a subtler case, the typo for the axial length can be in a second digit instead of a first (e.g., 20.87) which may still cause the determination engine 101 to produce a low confidence factor using data analytics.

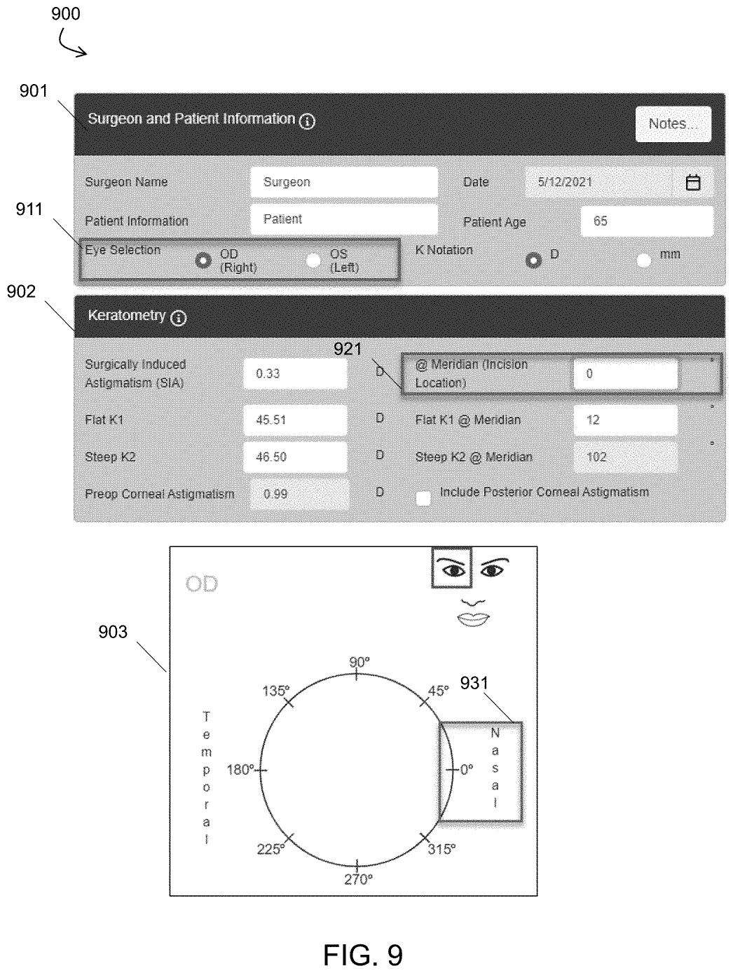

FIG. 9 depicts a user interface 900 according to one or more embodiments. The user interface 900 relates to possible action or thinking errors and includes sub-interfaces 901, 902, and 903. The user interface 900 can be a graphical user interface of the toric calculator showing likely/probable entry error for eye selection and meridian incision location in boxes 911 and 921. The user interface 900 demonstrates that the determination engine 101 can identify an action error slip (e.g., where the user either selected the wrong eye or forgot to change the eye selection) or a thinking error (e.g., where the user mistakenly entered a primary incision location that is on the Nasal side of the eye (see box 931)). In either case, the determination engine 101 can provide a low confidence factor for the eye selection and meridian incision location. Further, instances where the incision location varies from between temporal and superior through to those that are between nasal and inferior may result in a different confidence factor based on data analytics by the determination engine 101.

According to one or more embodiments, the determination engine 101 can provide an extension to an output selection guidance. The determination engine 101 can include one or more applications for data entry validation, while employing a similar approach in result selection guidance. For example, the determination engine 101 analyzes one or more lens models produced by a toric calculator, along with a residual astigmatism expressed as a cylinder and axis, in view of selected lenses and a variety of data entries.

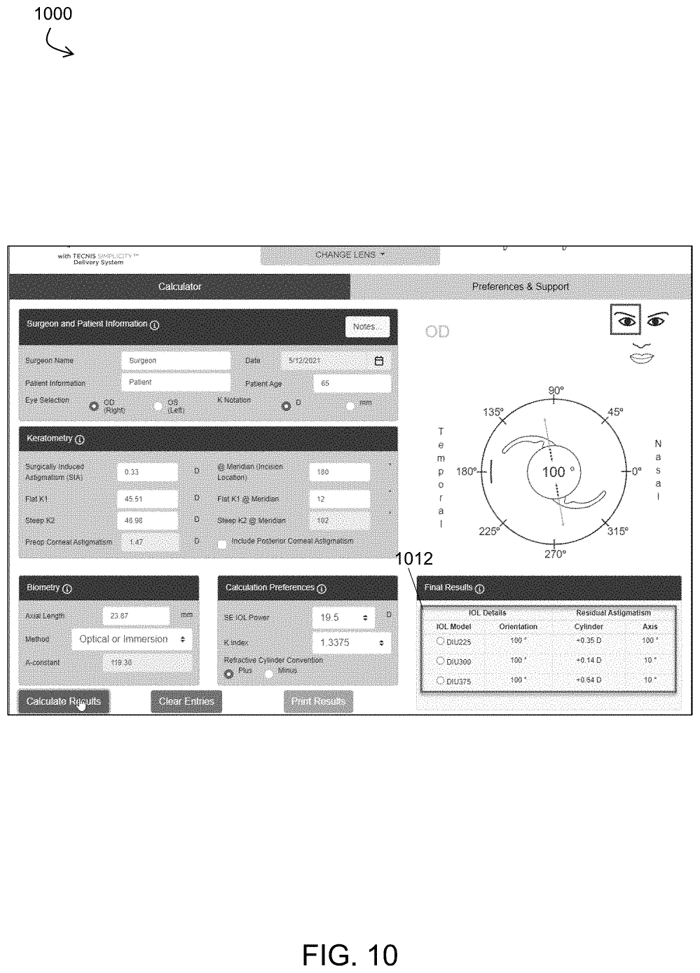

FIG. 10 depicts a user interface 1000 according to one or more embodiments. The user interface 1000 relates to a toric calculator results selection associated with final results for a toric calculator. That is, the user interface 1000 shows a graphical user interface presenting results of a calculation performed by the toric calculator on a set of data entries and selections. The user interface 1000 presents in box 1012 three lens models from which to choose. A user may interpret that the DIU300 lens is a best choice, since it will result in less residual astigmatism by 0.21 diopters. However, the DIU300 lens produces an axis flip (e.g., a shift of 90 degrees in the steep meridian). In turn, a less experienced user may be uncertain whether the DIU300 lens is truly the best choice.

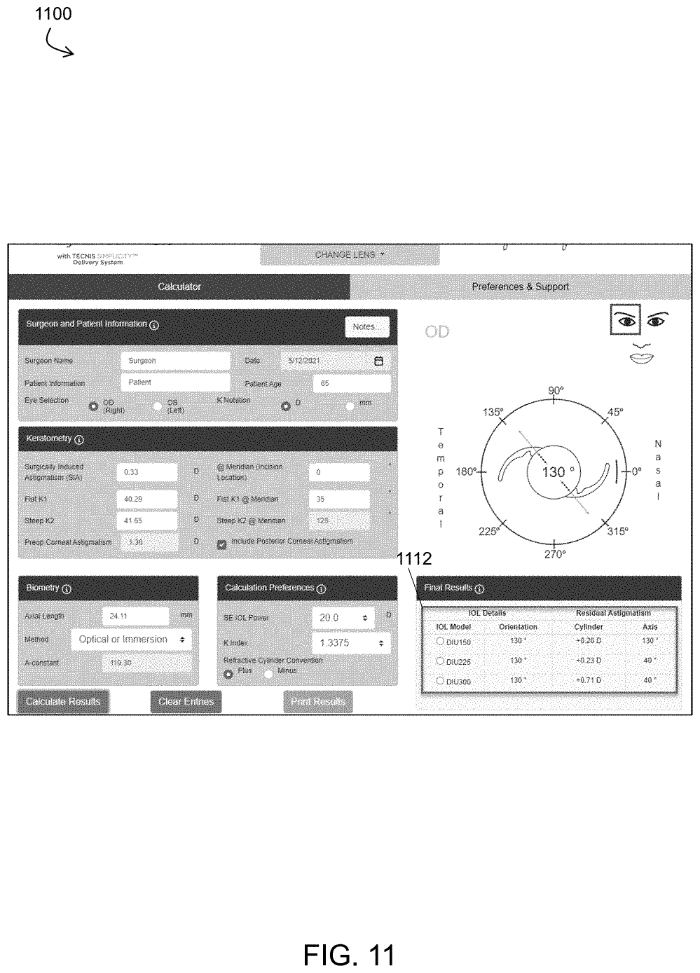

FIG. 11 depicts a user interface 1100 according to one or more embodiments. The user interface 1100 relates to an axis flip versus a residual astigmatism associated with final results for a toric calculator. That is, the user interface 1100 shows a graphical user interface presenting in box 1112 three lens models (from which to choose). Note that, in the user interface 1100 situation, the best choice is less obvious since the difference in residual astigmatism is very slight. Further, the user may choose the DIU150 lens model since it has nearly the best residual astigmatism and does not result in an axis flip.

Thus, in the FIGS. 10-11 scenarios, the determination engine 101 can provide one or more technical effects, advantages, and benefits by providing indications (e.g., the one or more interface elements and the one or more confidence factors) of what other users chose given a same or a similar circumstance. Further, the one or more confidence factors by the determination engine 101 can be based on whether a given lens model results in an axis flip and what a difference is between the residual astigmatisms for the lenses.

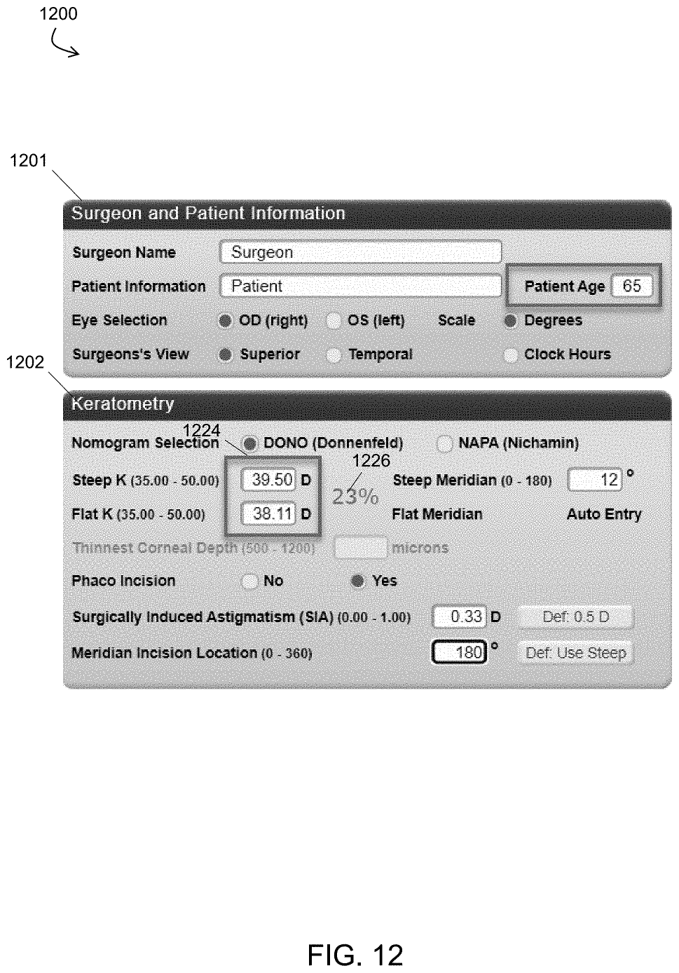

According to one or more embodiments, the determination engine 101 can provide representations of the one or more confidence factors with the user interface. Example of these representations include, but are not limited to, representing confidence as percentage or number, a rating from high to low, color-coding entry fields or entry labels, display of icons or symbols, discrete textual messaging for specific scenarios, and/or a combination thereof. Note that confidence can be expressed by the determination engine 101 for individual entries and/or selections, as well as for an overall summative confidence for a group of entries and/or selections. In addition, the determination engine 101 can utilize thresholds for when a confidence or guidance is or is not displayed. The FIGS. 12-16 are a few examples of how confidence might be employed within a typical SaMD and/or medical device user interface, but the principles could apply and be extended to other medical equipment and other distributed connected devices.

FIG. 12 depicts a user interface 1200 according to one or more embodiments. The user interface 1200 relates to confidence as a percentage or a number and includes sub-interfaces 1201 and 1202. More particularly, for a group of inputs/entries in box 1224, the determination engine 101 illustrates a confidence 1226 as a percentage. That is, the group of inputs/entries can be bounded with an interface element (e.g., a blue box) with a corresponding confidence factor for those inputs/entries displayed as a percentage. Note that the color of the box can be indicative of the level of confidence, as a reference, in addition to the percentage itself.

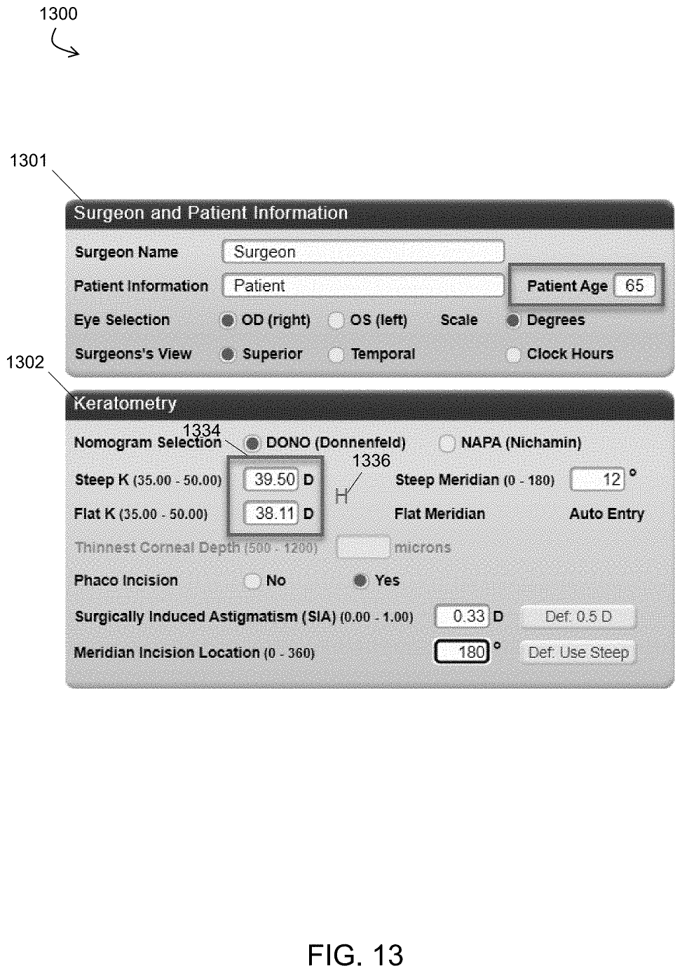

FIG. 13 depicts a user interface 1300 according to one or more embodiments. The user interface 1300 relates to confidence as a rating and includes sub-interfaces 1301 and 1302. More particularly, for a group of inputs/entries in box 1334, the determination engine 101 illustrates a confidence 1336 as a rating. As shown, the rating can be an “H” for “High”, with other rating including “M” for “Medium” and “L” for “Low”. The determination engine 101 can implement other descriptive scales as well.

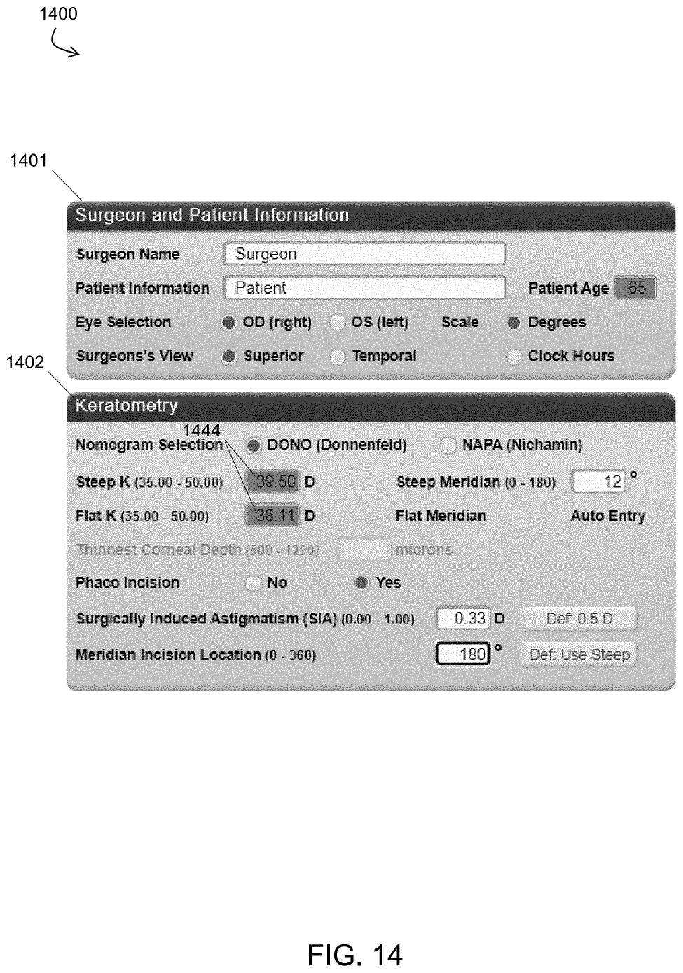

FIG. 14 depicts a user interface 1400 according to one or more embodiments. The user interface 1400 relates to confidence as a color-coding and includes sub-interfaces 1401 and 1402. More particularly, for a group of inputs/entries in box 1444, the determination engine 101 illustrates a confidence as a color-coded field. Note that color-coding by the determination engine 101 can be any discrete color scale, gradient, or combination thereof. In an example, a relatively low confidence can be presented by color-coding affected fields with a shade of red. In another example, confidence can be presented by color-coding labels for the affected fields or to color-code input/entry itself.

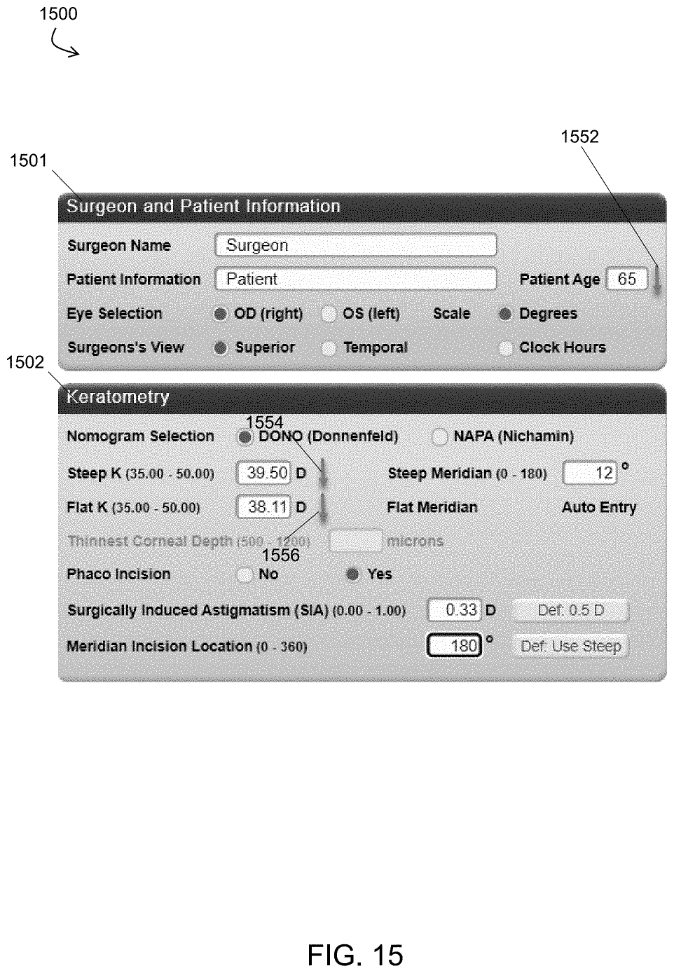

FIG. 15 depicts a user interface 1500 according to one or more embodiments. The user interface 1500 relates to confidence as an icon, a symbol, or a group of icons/symbols and includes sub-interfaces 1501 and 1502. More particularly, for each input/entry with a low confidence, the determination engine 101 illustrates a confidence as a symbol 1552, 1554, and 1556. For example, a low confidence is expressed by the determination engine 101 as a downward facing arrow.

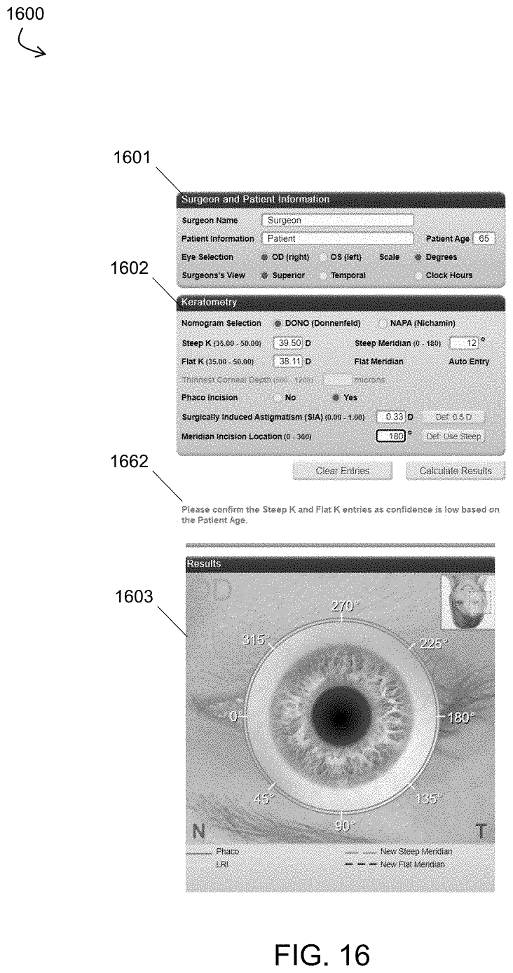

FIG. 16 depicts a user interface 1600 according to one or more embodiments. The user interface 1600 relates to confidence as discrete messaging and includes sub-interfaces 1601, 1602, and 1603. More particularly, for a group of inputs/entries with a low confidence, the determination engine 101 illustrates a confidence as a discrete message 1662. The discrete message 1662 can indicate to the user that the user should check the corresponding inputs.

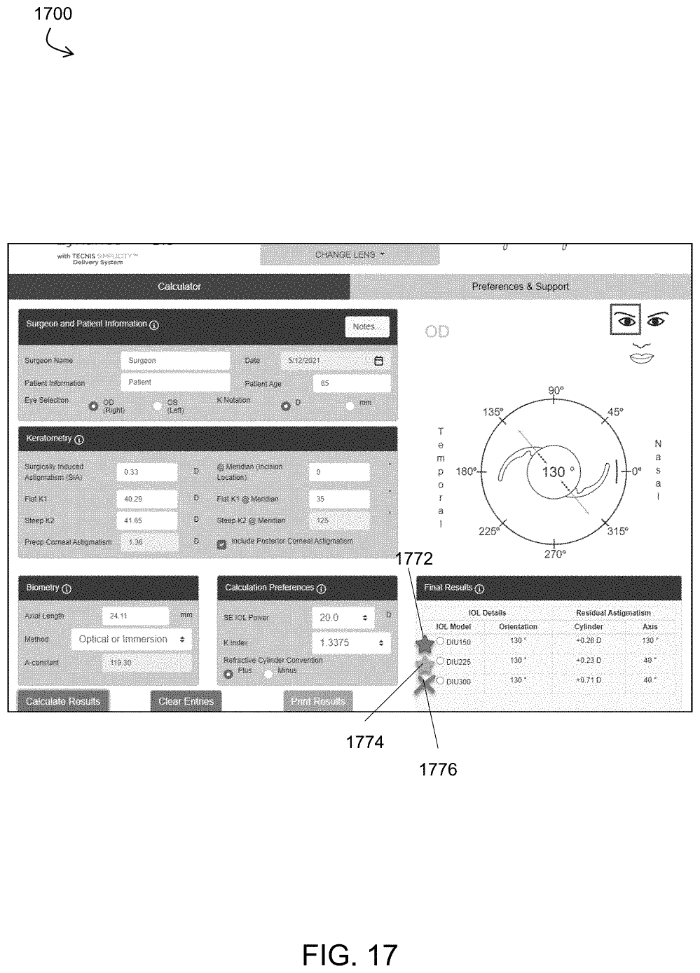

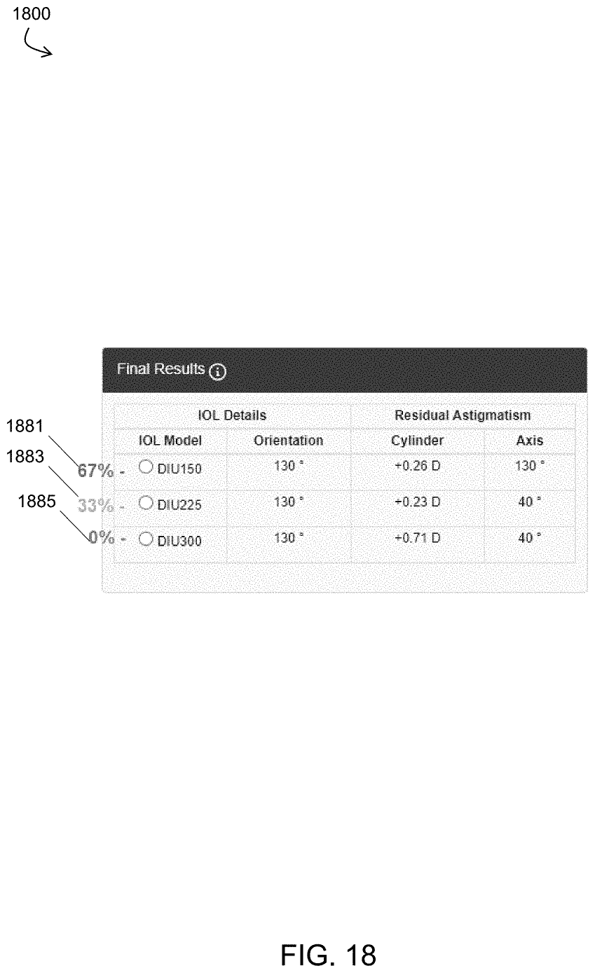

According to one or more embodiments, the determination engine 101 can provide representations for selection guidance within the user interface. The FIGS. 17-18 are examples of how selection guidance can be employed by the determination engine 101 and provided within a user interface of a SaMD application.

FIG. 17 depicts a user interface 1700 according to one or more embodiments. The user interface 1700 relates to selection guidance as a combination of color-coding and icons. The user interface 1700 illustrates a graphical user interface presenting a toric calculation and results thereof. In this regard, the user can be presented with up to three lens models choices based on orientation and residual astigmatism. The determination engine 101 provides star 1772 and 1774 next to lens models that have routinely been selected previously in similar circumstances and provides an “X” 1776 next to the lens model that has not. Additionally, the icons (e.g., the star 1772 and 1774 and the “X” 1776) can further be color-coded green, yellow, and red, respectively. Green can indicate a most frequently chosen, while red indicates a least frequently chosen.

FIG. 18 depicts a user interface 1800 according to one or more embodiments. The user interface 1800 relates to selection guidance as a color-coded percentage. In this regard, the user can be presented with up to three lens model choices based on orientation and residual astigmatism. The determination engine 101 provides percentages 1881, 1883, and 1885 with color-coding as a reference from most to least frequently selected (e.g., stoplight coloring, with green being the most and red being the least). The percentages can indicate a relative selection by previous users in similar circumstances.

According to one or more embodiments, the determination engine 101 can provide applications beyond SaMD applications, such as ophthalmic surgical equipment (e.g., phacoemulsification consoles and laser treatment systems) all medical equipment. According to one or more technical effects, advantages, or benefits, the determination engine 101 utilizing the embodiments herein can reduce data entry errors and provide guidance for end user selections, as well as enable improved accuracy of data entry and greater end-user satisfaction with selected outputs.

According to one or more embodiments, a method is provided. The method provides analytics driven user guidance based on data. The method is implemented by a determination engine stored on a memory as processor executable instructions. The method includes receiving one or more inputs associated with one or more fields of a user interface and aggregating the data associated with the one or more inputs. The method also includes evaluating the data and the one or more inputs to generate one or more interface elements or one or more confidence factors and providing the one or more interface elements or the one or more confidence factors, as the analytics driven user guidance, on the user interface to mitigate or prevent one or more inadvertent errors within the one or more inputs.

According to one or more embodiments or any of the embodiments herein, the user interface can include a graphical user interface of a limbal relaxing incisions calculator or a toric calculator.

According to one or more embodiments or any of the embodiments herein, the method can include applying the one or more confidence factors across one or more software models

According to one or more embodiments or any of the embodiments herein, the one or more software models can include a software as a medical device application or a medical device application.

According to one or more embodiments or any of the embodiments herein, the method can include accessing a uniform resource locator of the determination engine to cause operations of the determination engine to be presented in a web-based format with the user interface in a web browser.

According to one or more embodiments or any of the embodiments herein, the method can include generating and presenting the user interface by the determination engine to receive the one or more inputs.

According to one or more embodiments or any of the embodiments herein, the aggregating of the data and the evaluating of the data and the one or more inputs can occur in parallel.

According to one or more embodiments or any of the embodiments herein, the determination engine can operate in an offline mode where the aggregating of the data and the evaluating of the data and the one or more inputs occur after the determination engine receives the one or more inputs.

According to one or more embodiments or any of the embodiments herein, the determination engine can operate in an live mode where the aggregating of the data and the evaluating of the data and the one or more inputs occur while the medical professional is performing a procedure on a patient and providing the one or more inputs.

According to one or more embodiments or any of the embodiments herein, the one or more interface elements can include one or more of a geometric shape, highlighted area, grey-out area, modified texts, symbol, icon, and color coding.

According to one or more embodiments or any of the embodiments herein, the one or more confidence factors can include one or more of alpha-numeric values, shapes, objects, and colors selected from a range of no confidence to full confidence.

According to one or more embodiments or any of the embodiments herein, the method can include receiving user feedback that provides alternatives to the one or more user inputs. The method can also include evaluating the user feedback to provide one or more subsequent interface elements and confidence factors.

According to one or more embodiments or any of the embodiments herein, the determination engine can execute machine learning or artificial intelligence when aggregating of the data and evaluating of the data and the one or more inputs.

According to one or more embodiments or any of the embodiments herein, the one or more inadvertent errors can include of a tolerant input or a tolerant selection.

According to one or more embodiments or any of the embodiments herein, the tolerant input can include when at least one of the one or more inputs fall within an acceptable data entry range while representing a mistaken input.

According to one or more embodiments or any of the embodiments herein, the tolerant selection can include when at least one of the one or more inputs provide sub-optimal results.

According to one or more embodiments or any of the embodiments herein, the one or more inadvertent errors can include an action error or a thinking error.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Although features and elements are described above in particular combinations, one of ordinary skill in the art will appreciate that each feature or element can be used alone or in any combination with the other features and elements. In addition, the methods described herein may be implemented in a computer program, software, or firmware incorporated in a computer-readable medium for execution by a computer or processor. A computer readable medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Examples of computer-readable media include electrical signals (transmitted over wired or wireless connections) and computer-readable storage media. Examples of computer-readable storage media include, but are not limited to, a register, cache memory, semiconductor memory devices, magnetic media, internal hard disks, solid state drives (SSDs), removable disks, magneto-optical media, optical media, compact disks (CD), digital versatile disks (DVDs), a random access memory (RAM), a read-only memory (ROM), an erasable programmable read-only memory (EPROM or Flash memory), a static random access memory (SRAM), and a memory stick. A processor in association with software may be used to implement a radio frequency transceiver for use in a terminal, base station, or any host computer.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one more other features, integers, steps, operations, element components, and/or groups thereof.

The descriptions of the various embodiments herein have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method for analytics driven user guidance based on data, the method implemented by a determination engine stored on a memory as processor executable instructions, the method comprising:
receiving one or more inputs associated with one or more fields of a user interface;
aggregating the data associated with the one or more inputs;
evaluating the data and the one or more inputs to generate one or more interface elements or one or more confidence factors; and
providing the one or more interface elements or the one or more confidence factors, as the analytics driven user guidance, on the user interface to mitigate or prevent one or more inadvertent errors within the one or more inputs.

2. The method of claim 1, wherein the user interface comprises a graphical user interface of a limbal relaxing incisions calculator or a toric calculator.

3. The method of claim 1, further comprising:
applying the one or more confidence factors across one or more software models.

4. The method of claim 3, wherein the one or more software models comprise a software as a medical device application or a medical device application.

5. The method of claim 1, further comprising:
accessing a uniform resource locator of the determination engine to cause operations of the determination engine to be presented in a web-based format with the user interface in a web browser.

6. The method of claim 1, further comprising:
generating and presenting the user interface by the determination engine to receive the one or more inputs.

7. The method of claim 1, wherein the aggregating of the data and the evaluating of the data and the one or more inputs occur in parallel.

8. The method of claim 1, wherein the determination engine operates in an offline mode where the aggregating of the data and the evaluating of the data and the one or more inputs occur after the determination engine receives the one or more inputs.

9. The method of claim 1, wherein the determination engine operates in an live mode where the aggregating of the data and the evaluating of the data and the one or more inputs occur while the medical professional is performing a procedure on a patient and providing the one or more inputs.

10. The method of claim 1, wherein the one or more interface elements comprise one or more of a geometric shape, highlighted area, grey-out area, modified texts, symbol, icon, and color coding.

11. The method of claim 1, wherein the one or more confidence factors comprise one or more of alpha-numeric values, shapes, objects, and colors selected from a range of no confidence to full confidence.

12. The method of claim 1, further comprising:
receiving user feedback that provides alternatives to the one or more user inputs, and
evaluating the user feedback to provide one or more subsequent interface elements and confidence factors.

13. The method of claim 1, wherein the determination engine executes machine learning or artificial intelligence when aggregating of the data and evaluating of the data and the one or more inputs.

14. The method of claim 1, wherein the one or more inadvertent errors comprises of a tolerant input or a tolerant selection.

15. The method of claim 14, wherein the tolerant input comprises when at least one of the one or more inputs fall within an acceptable data entry range while representing a mistaken input.

16. The method of claim 14, wherein the tolerant selection comprises when at least one of the one or more inputs provide sub-optimal results.

17. The method of claim 1, wherein the one or more inadvertent errors comprises an action error or a thinking error.
